(19) United States
(12) Patent Application Publication
(10) Pub. No.: US 2025/0281403 A1
(43) Pub. Date: Sep. 11, 2025

(54) FORMULATIONS

(71) Applicant: UCB Biopharma Srl, Brussels (BE)

(72) Inventors: Sarah MARQUETTE, Brussels (BE); Claude PEERBOOM, Brussels (BE); Michaël Joseph Edouard BOONEN, Brussels (BE)

(21) Appl. No.: 18/703,675

(22) PCT Filed: Oct. 20, 2022
(86) PCT No.: PCT/EP2022/079185

§ 371 Date: Apr. 22, 2024

(30) Foreign Application Priority Data
Oct. 21, 2021 (GB) 2115127.9

(51) Int. Cl. A61K 9/08; A61K 47/18; A61K 47/22; A61K 47/26; C07K 16/40

(52) U.S. Cl. CPC A61K 9/08; A61K 47/183; A61K 47/22; A61K 47/26; C07K 16/40

(57) ABSTRACT

The invention relates to the field of pharmaceutical formulations. More particularly it is directed to liquid formulations comprising anti-TG2 antibodies and to methods of producing such formulations. The liquid formulations according to the invention are stable upon storage at a temperature from about 2 to 25° C. for an appropriate period of time.

### FIELD OF INVENTION

The invention relates to the field of pharmaceutical formulations. More particularly it is directed to liquid formulations comprising anti-TG2 antibodies and to methods of producing such formulations. The liquid formulations according to the invention are stable upon storage at a temperature from about 2 to 25° C. for an appropriate period of time.

### BACKGROUND OF THE INVENTION

Tissue transglutaminase (TG2) is an enzyme which forms crosslinks between proteins via epsilon (gamma-glutamyl) lysine bridges. Elevated expression of TG2 leads to aberrant protein cross-linking which has been associated with several pathologies including various types of tissue scarring, the formation of neurofibrillary tangles in several brain disorders and resistance to chemotherapy in some cancers. Various TG2 inhibitors, such as small molecules, silencing RNA or antibodies (e.g. WO2006100679, WO2012146901 or WO2013175229), have been disclosed for the possible treatment of TG2-mediated disorders.

Although antibodies directed to TG2 have been described in the literature, no stable formulations have been proposed so far.

When preparing a pharmaceutical composition comprising a bioactive protein, such as an antibody, said composition must be formulated in such a way that the protein is stable for an appropriate period of time. A loss in activity/stability of the protein may result from chemical or physical instabilities of the protein notably due to denaturation, aggregation or oxidation. The resulting products may thus be pharmaceutically unacceptable. Although the use of excipient(s) is known to increase the stability of a given protein, the stabilizing effects of these excipients is highly dependent of the nature of the excipients and of the bioactive protein itself.

There remains a need for formulations containing an anti-TG2 antibody as an active ingredient, at high concentration, wherein said formulations are stable for an appropriate period of time and suitable for use in injection, such as for intravenous or subcutaneous injection. Said formulations could be useful for administration in the treatment of TG2-mediated disorders or diseases.

### SUMMARY OF THE INVENTION

It is an object of the present invention to provide novel formulations containing an anti-TG2 antibody. More particularly, said formulations are stable liquid formulations containing an anti-TG2 antibody, preferably at high concentration. The invention also provides methods for preparing the liquid formulations according to the present invention. The liquid formulations herein described may be useful for administration in the treatment of TG2-mediated disorders or diseases.

In a first aspect, the invention provides a stable liquid formulation comprising or consisting of an anti-TG2 antibody, a buffer which keeps the pH at or about 5.0 to 6.0, an amino acid stabilizer, and optionally a polysorbate surfactant. In a preferred embodiment, the buffer is a histidine, and the amino acid is arginine or an arginine salt (such as arginine-HCl). In a further preferred embodiment, the buffer keeps the pH at or about 5.5 (such as 5.5±0.2). In a further preferred embodiment, the anti-TG2 antibody is in an amount of or of about 50 mg/mL to or to about 300 mg/mL. Preferably, the anti-TG2 antibody comprises a light chain variable region as defined in SEQ ID NO: 1 and a heavy chain variable region as defined in SEQ ID NO: 2.

In a second aspect, the invention provides a method for manufacturing a stable liquid formulation of an anti-TG2 antibody, comprising the steps of forming a mixture of anti-TG2 antibody, together with a buffer, an amino acid stabilizer, and optionally a polysorbate surfactant. In a preferred embodiment, the buffer is a histidine, and the amino acid is arginine or an arginine salt (such as arginine-HCl). In a further preferred embodiment, the buffer keeps the pH at or about 5.0 to 6.0 (such as 5.5±0.2). In a further preferred embodiment, the anti-TG2 antibody is in an amount of or of about 50 mg/mL to or to about 300 mg/mL. Preferably, the anti-TG2 antibody comprises a light chain variable region as defined in SEQ ID NO: 1 and a heavy chain variable region as defined in SEQ ID NO: 2.

In a third aspect, here is provided an article of manufacture for pharmaceutical or veterinary use, comprising a container comprising the stable liquid formulation according to the invention.

In a fourth aspect, the invention provides the stable liquid formulation according to the invention for use in therapy

In a fifth aspect, the invention provides a method for treating a disease or disorder by administering the stable liquid formulation according to the invention.

### Definitions

The term “about” means approximately or nearly, and in the context of a numerical value set forth herein preferably designates +/−10% around the numerical value recited or claimed.
When a range of values is recited or claimed, the range is intended to be inclusive of the recited values.
The term “anti-TG2 antibody”, as used herein, is intended to be an antibody molecule which binds the Tissue transglutaminase (TG2) protein, an enzyme which forms crosslinks between proteins via epsilon (gamma-glutamyl) lysine bridges. Examples of such antibodies are described in WO2013175229. Without any limitation, an anti-TG2 antibody that can be used according to the present invention comprises for instance a light chain variable region as defined in SEQ ID NO: 1 and a heavy chain variable region as defined in SEQ ID NO: 2.
The term “antibody” as used herein includes, but is not limited to, monoclonal antibodies, polyclonal antibodies and recombinant antibodies that are generated by recombinant technologies as known in the art. “Antibody” include antibodies of any species, in particular of mammalian species; such as human antibodies of any isotype, including IgG1, IgG2a, IgG2b, IgG3, IgG4, IgE, IgD and antibodies that are produced as dimers of this basic structure including IgGA1, IgGA2, or pentamers such as IgM and modified variants thereof; non-human primate antibodies, e.g. from chimpanzee, baboon, rhesus or cynomolgus monkey; rodent antibodies, e.g. from mouse, or rat; rabbit, goat or horse antibodies; camelid antibodies (e.g. from camels or llamas such as Nanobodies™) and derivatives thereof; antibodies of bird species such as chicken antibodies; or antibodies of fish species such as shark antibodies. The term “antibody” also refers to “chimeric” antibodies in which a first portion of at least one heavy and/or light chain antibody sequence is from a first species and a second portion of the heavy and/or light chain antibody sequence is from a second species. Chimeric antibodies of interest herein include “primatized” antibodies comprising variable domain antigen-binding sequences derived from a non-human primate (e.g. Old World Monkey, such as baboon, rhesus or cynomolgus monkey) and human constant region sequences. “Humanized” antibodies are chimeric antibodies that contain a sequence derived from non-human antibodies. For the most part, humanized antibodies are human antibodies (recipient antibody) in which residues from a hypervariable region of the recipient are replaced by residues from a hypervariable region [or complementarity determining region (CDR)] of a non-human species (donor antibody) such as mouse, rat, rabbit, chicken or non-human primate, having the desired specificity, affinity, and activity. In most instances residues of the human (recipient) antibody outside of the CDR; i.e. in the framework region (FR), are additionally replaced by corresponding non-human residues. Furthermore, humanized antibodies may comprise residues that are not found in the recipient antibody or in the donor antibody. These modifications are made to further refine antibody properties. Humanization reduces the immunogenicity of non-human antibodies in humans, thus facilitating the application of antibodies to the treatment of human disease. Humanized antibodies and several different technologies to generate them are well known in the art. The term “antibody” also refers to human antibodies, which can be generated as an alternative to humanization. For example, it is possible to produce transgenic animals (e.g., mice) that are capable, upon immunization, of producing a full repertoire of human antibodies in the absence of production of endogenous murine antibodies. Other methods for obtaining human antibodies/antibody fragments in vitro are based on display technologies such as phage display or ribosome display technology, wherein recombinant DNA libraries are used that are either generated at least in part artificially or from immunoglobulin variable (V) domain gene repertoires of donors. Phage and ribosome display technologies for generating human antibodies are well known in the art. Human antibodies may also be generated from isolated human B cells that are ex vivo immunized with an antigen of interest and subsequently fused to generate hybridomas which can then be screened for the optimal human antibody. The term “antibody” refers to both glycosylated and aglycosylated antibodies. Furthermore, the term “antibody” as used herein not only refers to full-length antibodies, but also refers to antibody fragments, more particularly to antigen-binding fragments thereof. A fragment of an antibody comprises at least one heavy or light chain immunoglobulin domain as known in the art and binds to one or more antigen(s). Examples of antibody fragments according to the invention include a Fab, modified Fab, Fab′, modified Fab′, F(ab′)2, Fv, Fab-Fv, Fab-dsFv, Fab-Fv-Fv, scFv and Bis-scFv fragment. Said fragment can also be a diabody, tribody, triabody, tetrabody, minibody, single domain antibody (dAb) such as sdAb, VL, VH, VHH or camelid antibody (e.g. from camels or llamas such as a Nanobody™) and VNAR fragment. An antigen-binding fragment according to the invention can also comprise a Fab linked to one or two scFvs or dsscFvs, each scFv or dsscFv binding the same or a different target (e.g., one scFv or dsscFv binding a therapeutic target and one scFv or dsscFv that increases half-life by binding, for instance, albumin). Exemplary of such antibody fragments are FabdsscFv (also referred to as BYbe®) or Fab-(dsscFv)2 (also referred to as TrYbe®, see WO2015/197772 for instance). Antibody molecules as defined above, including antigen-binding fragments thereof, are known in the art.
The term “stability”, as used herein, refers to the physical, chemical, and conformational stability of the anti-TG2 antibody formulations according to the present invention (and including maintenance of biological potency). Instability of said antibody formulation may be caused by chemical degradation or aggregation of the antibody to form higher order polymers, deglycosylation, modification of glycosylation, oxidation or any other structural modification that reduces at least one biological activity of the antibody.
The term “stable formulation” refers to a formulation in which the protein of interest (herein an anti-TG2 antibody) essentially retains its physical, chemical and biological properties upon storage. In order to measure the stability of an antibody in a formulation, various analytical methods are well within the knowledge of the skilled person (see some examples in the example section). Stability is typically assessed at a selected temperature (for instance ≤−60° C., 2-8° C., 25° C., 35° C. or more) for a selected time period (e.g. 3 months, 6 months, 12 months or more). As an antibody, once formulated, is typically stored in the fridge (typically 2-8° C.) or at room temperature (typically 15-25° C.) before being administered to a patient, it is important that said formulated antibody is stable over time at least at a temperature range of 2 to 25° C., as herein shown for example at 2-8° C. and 25° C. Various values can be used to conclude about stability over a given time period, such as (and not limited to): 1) not less than 90% of monomeric form of the antibody, 2) no more than 10% of alteration of the monomeric form of the antibody (in comparison of the initial data), 3) no more than 5% of High Molecular Weight Species (HMW or HMWS; also herein referred to as aggregates), or 4) no more than +/−0.2 unit variation of the pH (in comparison with the initial data).
The term “buffer”, as used herein, refers to solutions of compounds that are known to be safe in formulations for pharmaceutical or veterinary use and that have the effect of maintaining or controlling the pH of the formulation in the pH range desired for the formulation. Acceptable buffers for controlling pH at a moderately acidic pH to a moderately basic pH include, but are not limited to, phosphate, acetate, citrate, arginine, histidine and TRIS (2-amino-2-hydroxymethyl-1,3, -propanediol, the term includes any pharmacologically acceptable salt thereof) buffers.
The term “surfactant”, as used herein, refers to a soluble compound that can be used notably to increase the water solubility of hydrophobic, oily substances or otherwise increase the miscibility of two substances with different hydrophobicities. For this reason, these polymers are commonly used in industrial applications, cosmetics, and pharmaceuticals. They are also used as model systems for drug delivery applications, notably in order to modify the absorption of the drug or its delivery to the target tissues. Well known surfactants include polysorbates (polyoxyethylene derivatives; Tween) as well as poloxamers (i.e. copolymers based on ethylene oxide and propylene oxide, also known as Pluronics®).
The term “stabilizing agent”, “stabilizer” or “isotonicity agent”, as used herein, is a compound that is physiologically tolerated and imparts a suitable stability/tonicity to a formulation. It prevents notably the net flow of water across cell membranes that are in contact with the formulation. Compounds such as glycerin, are commonly used for such purposes. Other suitable stability agents include, but are not limited to, amino acids or proteins (e.g. glycine or albumin), salts (e.g. sodium chloride), and sugars (e.g. dextrose, mannitol, sucrose and lactose).
The term “vial” or “container”, as used herein, refers broadly to a reservoir suitable for retaining the anti-TG2 antibody formulation in liquid form. Examples of a vial that can be used in the present invention include an ampoule, a tube, a bottle, a syringe (such as a pre-filled syringe), cartridges, or other such reservoir suitable for delivery of the anti-TG2 antibody formulation to the patient via injection, preferably via intravenous or subcutaneous injection.
The term “solvent”, as used herein, refers to a liquid solvent either aqueous or nonaqueous. The selection of the solvent depends notably on the solubility of the drug compound on said solvent and on the mode of administration. Aqueous solvent may consist solely of water, or may consist of water plus one or more miscible solvents, and may contain dissolved solutes such as sugars, buffers, salts or other excipients. The more commonly used non-aqueous solvents are the short-chain organic alcohols, such as, methanol, ethanol, propanol, short-chain ketones, such as acetone, and poly alcohols, such as glycerol. According to the present invention, the preferred solvent is an aqueous solvent such as water or a saline solvent.
The term “treating” or “treatment” of a disease state includes: (i) inhibiting the disease state, i.e. arresting the development of the disease state or its clinical symptoms, or (ii) relieving the disease state, i.e. causing temporary or permanent regression of the disease state or its clinical symptoms.
The term “preventing” or “prevention” of a disease state includes causing the clinical symptoms of the disease state not to develop in a subject that may be exposed to or predisposed to the disease state, but does not yet experience or display symptoms of the disease state.—In all the embodiments of the invention, “pharmaceutical composition” can also be referred to as “stable pharmaceutical composition” without any differentiation.

### DETAILED DESCRIPTION OF THE INVENTION

The invention is based on the combination of an arginine-based stabilizer and a histidine buffer, keeping the pH between 5.0 to 6.0 for preparing a suitable pharmaceutical composition for human use of an anti-TG2 antibody, preferably at high concentrations, without affecting the processability of the pharmaceutical composition and the long-term stability of the antibody. It is a finding from the inventors that the pharmaceutical compositions according to the invention are stable overtime, in particular when stored at about 2-25° C., as shown in the examples section at 2-8° C. and 25° C. The main object of the present invention is a stable liquid formulation comprising or consisting of an anti-TG2 antibody, a buffer keeping the pH between about 5.0 and about 6.0, and an amino acid stabilizer. In a preferred embodiment, the buffer is a histidine buffer, and the amino acid stabilizer selected from the group consisting of arginine or an arginine salt (such as arginine-HCl). The invention further provides a method for manufacturing any of the herein described stable liquid formulations of an anti-TG2 antibody, wherein the method comprises the steps of combining the anti-TG2 antibody, together with a buffer, an amino acid stabilizer and optionally a surfactant, such as a polysorbate surfactant. Said step is typically performed by buffer exchange according to conventional procedures. As an example, in order to prepare a suitable stable formulation, a given amount of an anti-TG2 antibody is buffer exchanged with a histidine buffer which keeps the pH at or about 5.0 to 6.0, and an amino acid stabilizer (preferably arginine or an arginine salt (such as arginine-HCl)). After buffer exchange, the formulation is filtered (final filtration). Depending on the target concentration for the antibody, the formulation can be concentrated between the step of buffer exchange and the final filtration. Should the formulation comprise a surfactant, it is preferably added after the concentration step, if any. Each of these compounds (i.e. the anti-TG2 antibody, the buffer, the amino acid stabilizer and optionally the surfactant) can be used according to the concentrations, pH, and/or ratios herein described. The resulting mixture is then dispensed into a container. Variations of this process will be recognized by one of ordinary skill in the art.

The invention also provides an article of manufacture, for pharmaceutical or veterinary use, comprising a container comprising any of the herein described stable liquid formulations, said formulations comprising or consisting of anti-TG2 antibody, a buffer, an amino acid stabilizer, and optionally a surfactant. Each of these compounds (i.e. the anti-TG2 antibody, the buffer, the amino acid stabilizer and optionally the surfactant) can be used according to the concentrations, pH, and/or ratios herein described.

Also described, a packaging material providing instructions for use.

Preferably, the anti-TG2 antibody to be used according to the invention as a whole comprises (see also Table A):
1) a light chain variable domain having the sequence as defined in SEQ ID NO: 1 and a heavy chain variable domain having the sequence as defined in SEQ ID NO: 2
2) a light chain variable domain having at least 80% identity or similarity, preferably 90% identity or similarity to the sequence as defined in SEQ ID NO: 1 and a heavy chain variable domain having at least 80% identity or similarity, preferably 90% identity or similarity to the sequence as defined in SEQ ID NO: 2.
3) a light chain having the sequence as defined in SEQ ID NO: 3 and a heavy chain having the sequence as defined in SEQ ID NO: 4; or
4) a light chain having at least 80% identity or similarity, preferably 90% identity or similarity to the sequence as defined in SEQ ID NO: 3 and a heavy chain having at least 80% identity or similarity, preferably 90% identity or similarity to the sequence as defined in SEQ ID NO: 4.

TABLE A
Anti-TG2 amino acid sequences

| SEQ | |
|---|---|
| ID | Amino acid sequence |
| 1 | DITMTQSPSSLSASVGDRVTITCKASQDINSYLTW |
| | FQQKPGKAPKILIYLVNRLVDGVPSRFSGSGSGQD |
| | YALTISSLQPEDFATYYCLQYDDFPYTFGQGTKVE |
| | IK |
| 2 | EVQLLESGGGLVQPGGSLRLSCAASGFTLSTHAMS |
| | WVRQAPGKGLEWVATISSGGRSTYYPDSVKGRFTI |
| | SRDNSKNTLYLQMNSLRAEDTAVYFCARLISTYWG |
| | QGTLVTVSS |
| 3 | DITMTQSPSSLSASVGDRVTITCKASQDINSYLTW |
| | FQQKPGKAPKILIYLVNRLVDGVPSRFSGSGSGQD |
| | YALTISSLQPEDFATYYCLQYDDFPYTFGQGTKVE |
| | IKRTVAAPSVFIFPPSDEQLKSGTASVVCLLNNFY |
| | PREAKVQWKVDNALQSGNSQESVTEQDSKDSTYSL |
| | SSTLTLSKADYEKHKVYACEVTHQGLSSPVTKSFN |
| | RGEC |
| 4 | EVQLLESGGGLVQPGGSLRLSCAASGFTLSTHAMS |
| | WVRQAPGKGLEWVATISSGGRSTYYPDSVKGRFTI |
| | SRDNSKNTLYLQMNSLRAEDTAVYFCARLISTYWG |
| | QGTLVTVSSASTKGPSVFPLAPCSRSTSESTAALG |
| | CLVKDYFPEPVTVSWNSGALTSGVHTFPAVLQSSG |
| | LYSLSSVVTVPSSSLGTKTYTCNVDHKPSNTKVDK |
| | RVESKYGPPCPPCPAPEFLGGPSVFLFPPKPKDTL |
| | MISRTPEVTCVVVDVSQEDPEVQFNWYVDGVEVHN |
| | AKTKPREEQFNSTYRVVSVLTVLHQDWLNGKEYKC |
| | KVSNKGLPSSIEKTISKAKGQPREPQVYTLPPSQE |
| | EMTKNQVSLTCLVKGFYPSDIAVEWESNGQPENNY |
| | KTTPPVLDSDGSFFLYSRLTVDKSRWQEGNVFSCS |
| | VMHEALHNHYTQKSLSLSLGK |

In the context of the invention as a whole, the amount of anti-TG2 antibody in the formulations is preferably from or from about 50 mg/mL to or to about 300 mg/mL, preferably from or from about 100 mg/mL to or to about 250 mg/mL, or even preferably from or from about 100 mg/mL to or from about 220 mg/mL such as 100, 105, 110, 115, 120, 125, 130, 135, 140, 145, 150, 155, 160, 165, 170, 175, 180, 185, 190, 195, 200, 210 or 220 mg/mL. Alternatively, the anti-TG2 antibody is preferably present in the protein formulation in an amount expressed in terms of weight per 100 mL (% w/v). In such a case, the anti-TG2 antibody comprised in the formulations according to the present invention as a whole can be present in an amount of about 5 to or to about 30% w/v, preferably in an amount of about 10 to or to about 25% w/v, or even preferably in an amount of about 10 to about 22% w/v such as 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0, 19.5, 20.0, 20.5, 21.0, 21.5 or 22.0% w/v. The anti-TG2 antibody may for instance comprise a light chain variable region as defined in SEQ ID NO: 1 and a heavy chain variable region as defined in SEQ ID NO: 2.

Preferable buffer according to the present invention as a whole is a histidine buffer (such as L-histidine) and keep the pH comprised between about 5.0 and about 6.0, preferably comprised between about 5.2 and about 5.8, such as 5.2, 5.3, 5.4, 5.5, 5.6, 5.7 and 5.8. Even more preferably the pH is at or about 5.5. In all the embodiments of the present invention, unless otherwise indicated, the pH value was measured at room temperature and it is preferably within ±0.1 or ±0.2 of the targeted pH unit (e.g. 5.5±0.1 or 5.5±0.2).

In the context of the invention as a whole, the buffer concentration is preferably at or about 10 to 100 mM. In a preferred embodiment, the concentration of the buffer is at or about 20 to or to about 80 or even preferably about 40 to about 60 mM, such as 40, 45, 50, 55 or 60 mM. Preferably, the concentration of the buffer is at or about 50 mM.

In the context of the invention as a whole, the amino acid stabilizer is selected from the group consisting of arginine or an arginine salt. Preferably, the arginine or arginine salt is L-arginine or L-arginine salt. Its concentration is preferably at or at about 100 mM to or to about 300 mM, preferably at or at about 110 to or to about 250 mM or even preferably at or at about 110 to or to about 200 mM, such as at or at about 110, 115, 120, 125, 130, 135, 140, 145, 150, 155, 160, 165, 170, 175, 180, 185, 190, 195 or 200 mM. Arginine, or arginine salts, also acts as a viscosity-reducing agent in the formulations according to the invention.

In the context of the invention as a whole, a surfactant can be optionally present. When present, the surfactant is preferably a polysorbate surfactant such as polysorbate 20 (PS20 also known as Tween® 20) or polysorbate 80 (PS80 also known as Tween® 80). Preferably the surfactant is present in the formulations in an amount of or of about 0.01 to or to about 5 mg/mL, more preferably of or of about 0.1 to or to about 1 mg/mL, more particularly of or of about 0.1 to or to about 0.5 mg/mL, such as 0.1, 0.15, 0.2, 0.25, 0.3, 0.35, 0.4, 0.45 or 0.5 mg/mL. Alternatively, the polysorbate surfactant is preferably present in the protein formulation in an amount expressed in terms of % weight per 100 mL (% w/v). In such a case, the polysorbate surfactant comprised in the formulations according to the present invention as a whole can be present in an amount of 0.001 to 0.5% w/v, preferably from 0.01 to 0.1% w/v, or even preferably from 0.01 to 0.05% w/v such as 0.01, 0.015, 0.02, 0.025, 0.03, 0.035, 0.04, 0.045 or 0.05% w/v.

In a preferred embodiment, the stable liquid formulation according to the present invention as a whole comprises or consists of an anti-TG2 antibody at or at about 50 to 300 mg/mL, about 10 to about 100 mM of histidine at pH about 5.5, about 100 to about 300 mM of arginine or an arginine salt, and optionally a surfactant (such as a polysorbate surfactant) at about 0.01 to 5 mg/mL. Alternatively, the stable liquid formulation in the present invention comprises or consists of an anti-TG2 antibody at about 5 to about 30% w/v, about 10 to about 100 mM of histidine at pH about 5.5, about 100 to about 300 mM of arginine or an arginine salt, and optionally 0.001 to 0.5% w/v of surfactant (such as a polysorbate surfactant).

As specific examples (but not limited to), herein are provided stable liquid formulations comprising or consisting of:
i) about 125 mg/mL of the anti-TG2 antibody, about 50 mM histidine buffer which keep the pH at or about 5.5, about 125 mM of Arginine-HCl, and optionally about 0.02-0.05% w/v of polysorbate,
ii) about 125 mg/mL of the anti-TG2 antibody, about 50 mM histidine buffer which keep the pH at or about 5.5, about 150 mM of Arginine-HCl, and optionally about 0.02-0.05% w/v of polysorbate,
iii) about 150 mg/mL of the anti-TG2 antibody, about 50 mM histidine buffer which keep the pH at or about 5.5, about 125 mM of Arginine-HCl, and optionally about 0.02-0.05% w/v of polysorbate,
iv) about 150 mg/mL of the anti-TG2 antibody, about 50 mM histidine buffer which keep the pH at or about 5.5, about 150 mM of Arginine-HCl, and optionally about 0.02-0.05% w/v of polysorbate,
v) about 175 mg/mL of the anti-TG2 antibody, about 50 mM histidine buffer which keep the pH at or about 5.5, about 125 mM of Arginine-HCl, and optionally about 0.02-0.05% w/v of polysorbate,
vi) about 175 mg/mL of the anti-TG2 antibody, about 50 mM histidine buffer which keep the pH at or about 5.5, about 150 mM of Arginine-HCl, and optionally about 0.02-0.05% w/v of polysorbate,
vii) about 200 mg/mL of the anti-TG2 antibody, about 50 mM histidine buffer which keep the pH at or about 5.5, about 125 mM of Arginine-HCl, and optionally about 0.02-0.05% w/v of polysorbate, or
viii) about 200 mg/mL of the anti-TG2 antibody, about 50 mM histidine buffer which keep the pH at or about 5.5, about 150 mM of Arginine-HCl, and optionally about 0.02-0.05% w/v of polysorbate,
   wherein the anti-TG2 antibody may for instance comprise a light chain variable region as defined in SEQ ID NO: 1 and a heavy chain variable region as defined in SEQ ID NO: 2.

Preferably the formulations of the invention retain at least 80% of the biological activity of the anti-TG2 antibody at the time of formulation and/or packaging over a period of at least 12 months (before the first use). The anti-TG2 antibody activity may be measured according to routine methods such as Elisa or cell-based assays.

Additional excipients for use within the pharmaceutical compositions according to the invention include, but are not limited to stabilizers, bulking agents, solubilising agents or combinations thereof.

The present invention also provides for a container comprising the pharmaceutical composition according to the invention. In particular, the container may be, without any limitations, a vial, an ampoule, a tube, a bottle, a cartridge or a syringe (such as a pre-filled syringe) comprising the pharmaceutical composition.

The container may be part of a kit-of-parts comprising one or more containers comprising the pharmaceutical compositions according to the invention and delivery devices such as a syringe, pre-filled syringe, an autoinjector, a needleless device, an implant or a patch, or other devices for parental administration and instructions of use.

The liquid formulations of the invention may be kept for at least about 12 months to about 36 months. Under preferred storage conditions, before the first use, the formulations are kept away from bright light (preferably in the dark), at temperature from about 2 to 25° C., e.g. at room temperature (at or about 25° C.) or at 2-8° C. (see following examples). Said formulations minimize the loss of active principle, i.e. an anti-TG2 antibody. It has also been found that said formulations are less prone to acidification and to formation of protein aggregates, while having an acceptable viscosity (preferably at about or below 30 cP).

The present invention provides stable liquid formulations of anti-TG2 antibody for use in therapy. For instance, the stable liquid formulations of anti-TG2 antibody, herein described, are suitable for pharmaceutical or veterinary use. The present invention also provides a method for treating a disease or disorder by administering stable liquid formulations of anti-TG2 antibody.

The stable liquid formulation comprising anti-TG2 antibody according to the present invention, can be administered for improving or for treating TG2-mediated disorders or diseases. Such TG2-mediated disorders or diseases can for instance be selected from the group consisting of Celiac disease, abnormal wound healing, scarring, keloids and hypertrophic scars, ocular scarring, inflammatory bowel disease, macular degeneration, Grave's ophtalmopathy, drug-induced ergotism, psoriasis, fibrotic diseases or fibrosis-related diseases, atherosclerosis, restenosis, inflammatory diseases, autoimmune diseases, neurodegenerative/neurological diseases (e.g. Huntington's Disease, Alzheimer's disease, Parkinson's disease, polyglutamine disease, spinobulbar muscular atrophy, dentatorubral-pallidoluysian atrophy, spinocerebellar ataxias 1, 2, 3, 6, 7 and 12, rubropallidal atrophy, spinocerebellar palsy), and/or cancer (e.g. glioblastomas such as glioblastoma in Li-Fraumeni syndrome and sporadic glioblastoma, malignant melanomas, pancreatic ductal adenocarcinomas, myeloid leukemia, acute myelogenous leukemia, myelodysplasia syndrome, myeloproliferative syndrome, gynaecological cancer, Kaposi's sarcoma, Hansen's disease, collagenous colitis).

The pharmaceutical composition according to the invention may be administered in a therapeutically effective amount. The term “therapeutically effective amount” as used herein refers to an amount of a therapeutic agent (i.e. an antibody) needed to treat, improve or prevent a TG2-mediated disorder or disease, or to exhibit a detectable therapeutic, pharmacological or preventative effect. For any antibody, the therapeutically effective amount can be estimated initially either in cell culture assays or in animal models, usually in rodents, rabbits, dogs, pigs or primates. The animal model may also be used to determine the appropriate concentration range and route of administration. Such information can then be used to determine useful doses and routes for administration in humans.

For the treatment of the above diseases and/or disorders, the appropriate dosage will vary depending upon, for example, the particular antibody to be employed, the subject treated, the mode of administration and the nature and severity of the condition being treated. In a particular embodiment, the pharmaceutical composition according to the invention is administered by intravenous or subcutaneous route. When administered via intravenous injection, it may be administered as a bolus injection or as a continuous infusion. Depending on the administration mode, the formulations herein described can be diluted in a solvent (such as NaCl) before use. The pharmaceutical composition according to any of the embodiments of the invention may also be administered by intramuscular injection. The pharmaceutical composition may be injected using a syringe, an injection device such as an autoinjector, a needleless device, a portable device, an implant and a patch.

The liquid pharmaceutical formulation of the invention is suitably administered to the patient at one time or over a series of treatments and may be administered to the patient at any time from diagnosis onwards; it may be administered as the sole treatment or in conjunction with other drugs or therapies useful in treating the conditions as described herein before.

The antibody may be the sole active ingredient in the liquid pharmaceutical formulation. Alternatively, the antibody may be administered in combination, e.g. simultaneously, sequentially or separately, with one or more other therapeutically active ingredients. Active ingredient as employed herein refers to an ingredient with a pharmacological effect, such as a therapeutic effect, at a relevant dose. In some embodiments the antibody in the pharmaceutical composition may be accompanied by other active ingredients including other antibodies or non-antibody ingredients, administered by the same or by a different route of administration, to treat other inflammatory or autoimmune diseases. In one embodiment, the subject is administered, simultaneously or in sequence (before and/or after) other antibody ingredients, such as anti-TNF antibodies or non-antibody ingredients such as small molecule drug molecules.

The following examples are provided to further illustrate the preparation of the formulations and compositions of the invention. The scope of the invention shall not be construed as merely consisting of the following examples.

### EXAMPLES

### Material

Anti-TG2 antibody: the anti-TG2 monoclonal antibody (mAb) that was used in the following examples comprised a light chain variable region as defined in SEQ ID NO: 1 and a heavy chain variable region as defined in SEQ ID NO: 2. It is named mAb1 in the following examples.

### Methods

Proteins concentration: Protein concentration was determined using the UV-Visible spectroscopic method, using the following equation: Concentration (mg/mL)=[(A280)/a×b]

where A280=Absorbance at 280 nm (AU); a=Mass Extinction Coefficient (1.34 mL mg⁻¹ cm⁻¹); b=Path length (1 cm)

Aggregations and fragmentations: Size Exclusion chromatography (SEC), such as Size Exclusion Ultra High-Performance Liquid Chromatography (SE UPLC), was used according to standard methods. The percentage peak area values for the main species, as well as the species that elute before and after the main peak, designated high molecular weight (HMVV) and low molecular weight (LMVV) species, respectively, were evaluated.

Acid and basic species: The presence of acid and basic species was evaluated using Isoelectric Capillary Electrophoresis (iCE) according to standard iCE methods (separating proteins based on differences in their charges). Typically, peaks eluting before the main peak are labelled as acidic species and those eluting post main peak are labelled as basic species.

pH was evaluated according to standard methods, using a pH meter equipped with a temperature compensating electrode.

Viscosity: Viscosity measurements were performed according to standard methods, using a Rheosense MicroVisc®.

### Example 1—Preliminary Screening

### 1.1. Selection of Buffer and Main Excipient

There was a need to identify a suitable formulation delivering the highest concentration possible of mAb1 (higher than 10%, possibly at least 15%, i.e. at least 150 mg/mL). As one of the main concerns when it comes to highly concentrated formulations of antibodies is viscosity, the preliminary screening focused on this aspect. Four different buffer types and various excipients were evaluated with regard to their effect on viscosity of mAb1. The mAb1 samples were buffer exchanged, according to standard methods, into the buffers listed in Table 1.

TABLE 1
Preformulations screening

| | Buffer | Targeted pH | Other excipients(s) |
|---|---|---|---|
| F1 | 150 mM Citric acid | 5.5 | — |
| F2 | 150 mM TriNa Citrate | 5.5 | — |
| F3 | 20 mM Histidine | 5.5 | 150 mM NaCl |
| F4 | 150 mM TriNa Citrate | 5.5 | 150 mM NaCl |
| F5 | 20 mM Histidine | 5.5 | 150 mM NaSulfate |
| F6 | 20 mM Histidine | 5.5 | 150 mM Na2HPO4 |
| F7 | 20 mM Histidine | 5.5 | 150 mM Nalodure |
| F8 | 20 mM Histidine | 5.5 | 150 mM Sodium thiocyanate |
| F9 | 40 mM Acetate | 5.0 | 110 mM Glycine |
| F10 | 40 mM Acetate | 5.0 | 330 mM Glycine |
| F11 | 80 mM Acetate | 5.0 | 110 mM Glycine |
| F12 | 80 mM Acetate | 5.0 | 330 mM Glycine |
| F13 | 20 mM Histidine | 5.5 | 150 mM Arginine + 150 mM citric acid |
| F14 | 20 mM Histidine | 5.5 | 150 mM Arginine + 150 mM succinic acid |
| F15 | 20 mM Histidine | 5.5 | 150 mM Arginine + 150 mM glutamic acid |
| F16 | 20 mM Histidine | 5.5 | 150 mM Arginine + 150 mM aspartic acid |
| F17 | 20 mM Histidine | 5.5 | 150 mM Histidine + 150 mM citric acid |
| F18 | 20 mM Histidine | 5.5 | 150 mM Histidine + 150 mM succinic acid |
| F19 | 20 mM Histidine | 5.5 | 150 mM Na Benensulfonate |
| F20 | 20 mM Histidine | 5.5 | 150 mM Na Toluenesulfonate |
| F21 | 20 mM Histidine | 5.5 | 150 mM Na Cophorsulfonate |
| F22 | 20 mM Histidine | 5.5 | 150 mM Lysine Cophorsulfonic acid |
| F23 | 20 mM Histidine | 5.5 | 150 mM Arginine Cophorsulfonic acid |

The different formulations were analysed with regards to viscosity and pH at the highest mAb1 concentration reached for each formulation (See Table 2)

TABLE 2
Effect of the various preformulations on viscosity and pH

| SAMPLE | Mab1 (mg/mL) | Viscosity (cP) | Measured pH |
|---|---|---|---|
| 20 mM Histidine | 83 | 3 | 5.63 |
| pH 5.5 (control) | 124 | 8 | 5.63 |
| | 157 | 21 | 5.63 |
| | 194 | 55 | 5.63 |
| | 223 | 85 | 5.63 |
| F1 | 116 | 6 | 2.28 |
| F2 | 230 | 13 | 7.70 |
| F3 | 235 | 14 | 5.74 |
| F4 | 218 | 18 | 7.57 |
| F5 | 216 | 10 | 5.92 |
| F6 | 239 | 21 | 7.73 |
| F7 | 260 | 18 | 5.79 |
| F8 | 270 | 18 | 5.76 |
| F9 | 234 | 53 | 5.19 |
| F10 | 228 | 18 | 5.19 |
| F11 | 230 | 22 | 5.10 |
| F12 | 227 | 18 | 5.12 |
| F13 | 249 | 40 | 3.65 |
| F14 | 220 | 12 | 4.47 |
| F15 | 247 | 25 | 5.71 |
| F16 | 274 | 35 | 5.13 |
| F17 | 215 | 25 | 3.74 |
| F18 | 256 | 46 | 4.64 |
| F19 | 211 | 25 | 5.72 |
| F20 | 176 | 10 | 5.74 |
| F21 | 253 | 50 | 6.15 |
| F22 | 226 | 23 | 5.27 |
| F23 | 200 | 12 | 5.81 |

Based on the preliminary results, formulations F3, F7, F10, F14 and F16 were further evaluated at different mAb1 concentrations. Indeed, a mAb1 concentration of above 220 mg/ml was reached while keeping a viscosity under 20 cP for most of these selected formulations. Although F16 had a viscosity of 35 cP, an exceptional mAb1 concentration of above 270 mg/ml was reached. Most of the preselected formulations were based on histidine buffer, pH 5.5. The behaviour of the selected formulations (from a viscosity and pH viewpoint) was assessed at decreasing concentrations, as reported in Table 3.

TABLE 3
Effect of mAb1 concentration on viscosity and pH

| | Mab1 concentration | Viscosity | |
|---|---|---|---|
| SAMPLE | (mg/mL) | (cP) | Measured pH |
| F3 | 253 | 51 | 5.57 |
| | 214 | 22 | 5.57 |
| | 186 | 12 | 5.57 |
| | 158 | 6 | 5.57 |
| | 108 | 3 | 5.57 |
| F7 | 251 | 52 | 5.56 |
| | 222 | 25 | 5.56 |
| | 194 | 13 | 5.56 |
| | 158 | 9 | 5.56 |
| | 108 | 3 | 5.56 |
| F10 | 238 | 74 | 5.02 |
| | 201 | 32 | 5.02 |
| | 160 | 11 | 5.02 |
| | 130 | 10 | 5.02 |
| | 88 | 3 | 5.02 |
| F14 | 300 | 71 | 4.48 |
| | 206 | 12 | 4.48 |
| | 114 | 3 | 4.48 |
| | 60 | 3 | 4.48 |
| F16 | 290 | 94 | 5.38 |
| | 240 | 35 | 5.38 |
| | 202 | 12 | 5.38 |
| | 160 | 12 | 5.38 |
| | 115 | 4 | 5.38 |
| Control | 234 | 56 | 5.51 |
| (50 mM His, | 210 | 36 | 5.51 |
| 250 mM Glycine) | 182 | 15 | 5.51 |
| | 148 | 8 | 5.51 |
| | 104 | 5 | 5.51 |

Based on the preliminary study, it appeared that the most promising formulations were the ones comprising histidine as a buffer and arginine (F14 and F16).

### Example 2—Arginine Salts Comparison as Viscosity Reducer

The preformulation work from Example 1 highlighted that formulations comprising histidine as a buffer and arginine were the most promising in order to formulate mAb1 at high concentrations while keeping acceptable viscosity (below 20 cP at or about 20% mAb1 in the formulation). New formulations based on histidine as a buffer and arginine salt as viscosity reducer and optionally other excipients were prepared as per Table 4.

TABLE 4
formulations for example 2

| Formulation # | Buffer | Main excipient | Surfactant |
|---|---|---|---|
| F1 (control) | 50 mM Histidine, pH 5.5 | 250 mM Glycine | N/A |
| F2 | 50 mM Histidine, pH 5.5 | 150 mM Arginine/Aspartate | N/A |
| F3 | 50 mM Histidine, pH 5.5 | 150 mM Arginine/Aspartate | PS80 |
| F5 | 50 mM Histidine, pH 5.5 | 150 mM Arginine/75 mM Succinate | PS80 |
| F6 | 50 mM Histidine, pH 5.5 | 150 mM Arginine/Glutamate | PS80 |
| F8 | 50 mM Histidine, pH 5.5 | 150 mM Arginine/50 mM Citrate | PS80 |
| F9 | 50 mM Histidine, pH 5.5 | 150 mM Arginine/Metha Sulfonate | PS80 |
| F10 | 50 mM Histidine, pH 5.5 | 150 mM Arginine/Benzene Sulfonate | PS80 |
| F11 | 50 mM Histidine, pH 5.5 | 150 mM Arginine/Glucuronate | PS80 |
| F12 | 50 mM Histidine, pH 5.5 | 150 mM Arginine/Hipurate | PS80 |
| F13 | 50 mM Histidine, pH 5.5 | 150 mM Arginine/Acetate | PS80 |
| F14 | 50 mM Histidine, pH 5.5 | 150 mM Arginine/75 mM Acetate | PS80 |
| F15 | 50 mM Histidine, pH 5.5 | 150 mM Arginine HCl | N/A |

The different formulations were analysed with regards to viscosity and pH at the highest mAb1 concentration reached for each formulation (See Table 5)

TABLE 5
effect of the various formulations on viscosity and pH

| | Viscosity (cp) | Viscosity (cp) | Viscosity (cp) |
|---|---|---|---|
| | mAb1 = | mAb1 = | mAb1 = |
| Formulation # | 150 mg/mL | 200 mg/mL | 240 mg/mL |
| F1 (control) | 8.6 | 31.3 | 37.5 |
| F2 | 5.5 | 12.2 | 29.5 |
| F3 | 5.5 | 12.7 | 33.8 |
| F5 | 5.3 | 12.7 | 27.5 |
| F6 | 5.6 | 12.0 | 34.5 |
| F8 | 5.4 | 16.5 | 24.4 |
| F9 | 5.4 | 11.5 | 27.3 |
| F10 | 5.2 | 13.3 | — |
| F11 | 6.1 | 14.2 | 33.1 |
| F12 | 3.6 | 6.6 | 12.9 |
| F13 | 5.6 | 12.7 | 29.0 |
| F14 | 8.4 | 22.8 | 53.6 |
| F15 | 5.2 | 14.1 | 31.4 |

As F1 and F14 had higher viscosities compared to the other formulations comprising arginine, they were not further considered. Although F12 had a much lower viscosity, it was not considered either as comprised a rare excipient. The other formulations had comparable viscosity. However, as the viscosity of the simpler formulation F15 was comparable to more sophisticated ones, it was selected for further stability studies.

### Example 3—Long Term (12 Months) Stability Study on Selected Formulations

Based on the above example, seven formulations have been chosen for long term studies:
F1: 100 mg/mL of mAb1, 50 mM histidine, 125 mM arginine-HCl, pH5.5, 0.03% PS80;
F2: 125 mg/mL of mAb1, 50 mM histidine, 125 mM arginine-HCl, pH5.5, 0.03% PS80;
F3: 150 mg/mL of mAb1, 50 mM histidine, 125 mM arginine-HCl, pH5.5, 0.03% PS80;
F4: 175 mg/mL of mAb1, 50 mM histidine, 125 mM arginine-HCl, pH5.5, 0.03% PS80;
F5: 200 mg/mL of mAb1, 50 mM histidine, 125 mM arginine-HCl, pH5.5, 0.03% PS80;
F6: 150 mg/mL of mAb1, 50 mM histidine, 125 mM arginine-HCl, pH5.5;
F7: 100 mg/ml of mAb1, 50 mM histidine, 250 mM glycine, pH5.5 (control formulation).

The long-term stability (tested at 5° C., 25° C. and 40° C.) of the different formulations were analysed with regards to pH, protein concentration, charge variants (via iCE3), aggregations and fragmentations (via SE UPLC).

HMWS (see Tables 6 to 8): HMWS % increase faster with increase of the mAb1 concentration in accelerated conditions (25° C. and 40° C.). No effect of the surfactant (PS80) was observed (see F3 vs F6). In overall, at similar mAb1 concentration, there was a better stability in the newly identified formulation (F1) vs the control formulation (F7). At 5° C. and 25° C., the stability is comparable between the 150 mg/mL formulation (i.e. 15%; F3) and the control formulation (F7). There are no differences at 40° C.

LMWS (see Tables 6 to 8): LMWS % decrease with mAb1 concentration. A small increase of LMWS was observed at 5 and 25° C. As for HMWS species, no effect of the surfactant (PS80) was identified (see F3 vs F6).

Monomer (see Tables 6 to 8): Stability was comparable for the F1 formulation vs the F7 formulation (i.e. at comparable concentration) at 5° C. and 25° C. At 5° C. and 25° C., comparable stability was also observed between the F3 formulation (15% mAb1) and the control formulation (F7, at 10% mAb).

Main peak, APG and BPG (see Tables 6 to 8): No effect of the surfactant (PS80) was observed (see F3 vs F6) with regards to main peak, APG and BPG. There was a higher APG % in the control formulation than in F1 (same mAb1 concentration in both), and a lower BPG %, at 25° C. and 40° C. In overall, there was a higher level of main peak with F1 compared to F7 at 5 and 25° C. (no observed difference at 40° C.).

Viscosity (see Table 9) Viscosity increased with concentration of mAb1 in the formulation, as expected. However, it was possible to maintained it at no more than about 20 cP for the most concentrated formulation. The preferred formulation, at 15%, had a viscosity of about 6 cP.

TABLE 6
long term stability data at 5° C.

| | 5° C. | | | | | | | | |
|---|---|---|---|---|---|---|---|---|---|
| | | | | | | | SE UPLC | | |
| | | | A280 | iCE3 | | | HMWS | Mono | LMWS |
| | Weeks | pH | (mg/mL) | APG | Main | BPG | (%) | (%) | (%) |
| F1 | 0 | 5.51 | 100.9 | 34.7 | 54.0 | 11.3 | 1.53 | 97.69 | 0.78 |
| (10%) | 4 | 5.50 | 107.3 | 34.4 | 54.6 | 11.0 | 1.67 | 97.53 | 0.80 |
| | 8 | 5.45 | 107.3 | 34.2 | 54.6 | 11.2 | 1.79 | 97.40 | 0.81 |
| | 13 | | | 33.1 | 55.4 | 11.5 | 1.88 | 97.09 | 1.03 |
| | 26 | 5.47 | 102.9 | 33.9 | 55.1 | 11.0 | 2.04 | 97.03 | 0.93 |
| | 42 | 5.52 | 105.3 | 34.1 | 55.3 | 10.7 | 2.11 | 96.99 | 0.91 |
| | 52 | 5.52 | 103.3 | 34.7 | 53.3 | 12.0 | 2.11 | 96.86 | 1.03 |
| F2 | 0 | 5.51 | 127.6 | 35.4 | 53.6 | 11.0 | 1.63 | 97.59 | 0.78 |
| (12.5%) | 4 | 5.49 | 133.8 | 34.1 | 54.8 | 11.2 | 1.78 | 97.45 | 0.77 |
| | 8 | 5.45 | 130.5 | 34.3 | 54.5 | 11.2 | 1.92 | 97.30 | 0.78 |
| | 13 | | | 33.5 | 56.5 | 10.0 | 2.01 | 97.04 | 0.95 |
| | 26 | 5.47 | 128.2 | 33.7 | 55.5 | 10.7 | 2.23 | 96.82 | 0.95 |
| | 42 | 5.51 | 129.7 | 33.9 | 54.7 | 11.3 | 2.30 | 96.79 | 0.91 |
| | 52 | 5.47 | 129.8 | 34.1 | 54.0 | 11.8 | 2.33 | 96.65 | 1.02 |
| F3 | 0 | 5.51 | 152.3 | 34.7 | 54.4 | 10.9 | 1.76 | 97.47 | 0.77 |
| (15%) | 4 | 5.49 | 158.4 | 34.1 | 54.6 | 11.3 | 1.90 | 97.34 | 0.76 |
| | 8 | 5.44 | 156.8 | 34.8 | 53.9 | 11.3 | 2.08 | 97.13 | 0.79 |
| | 13 | | | 33.0 | 55.3 | 11.7 | 2.15 | 96.90 | 0.95 |
| | 26 | 5.46 | 150.7 | 34.1 | 54.9 | 11.0 | 2.39 | 96.70 | 0.92 |
| | 42 | 5.49 | 154.0 | 34.2 | 55.4 | 10.4 | 2.46 | 96.61 | 0.93 |
| | 52 | 5.46 | 157.1 | 34.0 | 53.9 | 12.1 | 2.45 | 96.55 | 1.00 |
| F4 | 0 | 5.50 | 178.1 | 34.9 | 54.0 | 11.1 | 1.88 | 97.37 | 0.75 |
| (17.5%) | 4 | 5.49 | 179.3 | 34.8 | 53.8 | 11.4 | 2.02 | 97.21 | 0.76 |
| | 8 | 5.44 | 182.5 | 34.5 | 54.4 | 11.1 | 2.21 | 97.01 | 0.78 |
| | 13 | | | 33.9 | 55.8 | 10.3 | 2.29 | 96.75 | 0.95 |
| | 26 | 5.46 | 179.6 | 33.5 | 55.2 | 11.4 | 2.61 | 96.47 | 0.92 |
| | 42 | 5.50 | 178.9 | 34.8 | 53.6 | 11.6 | 2.67 | 96.44 | 0.89 |
| | 52 | 5.45 | 186.9 | 33.9 | 54.4 | 11.7 | 2.01 | 96.84 | 1.15 |
| F5 | 0 | 5.50 | 207.5 | 35.5 | 53.7 | 10.8 | 2.00 | 97.26 | 0.74 |
| (20%) | 4 | 5.48 | 215.2 | 34.7 | 55.2 | 10.2 | 2.17 | 97.10 | 0.73 |
| | 8 | 5.45 | 214.5 | 34.2 | 55.9 | 9.9 | 2.38 | 96.86 | 0.75 |
| | 13 | | | 33.4 | 55.9 | 10.7 | 2.48 | 96.64 | 0.88 |
| | 26 | 5.45 | 211.3 | 33.6 | 54.9 | 11.5 | 2.87 | 96.23 | 0.90 |
| | 42 | 5.50 | 211.8 | 34.4 | 54.5 | 11.1 | 2.93 | 96.12 | 0.95 |
| | 52 | 5.45 | 221.8 | 34.2 | 53.8 | 12.0 | 2.99 | 96.07 | 0.95 |
| F6 | 0 | 5.52 | 151.9 | 34.3 | 54.3 | 11.4 | 1.71 | 97.53 | 0.76 |
| (15%) | 4 | 5.48 | 157.5 | 34.6 | 54.4 | 11.0 | 1.89 | 97.38 | 0.74 |
| | 8 | 5.44 | 153.8 | 34.7 | 54.2 | 11.1 | 2.07 | 97.15 | 0.78 |
| | 13 | | | 33.8 | 56.0 | 10.2 | 2.13 | 96.97 | 0.90 |
| | 26 | 5.45 | 150.5 | 33.8 | 54.8 | 11.4 | 2.39 | 96.74 | 0.88 |
| | 42 | 5.48 | 158.0 | 34.4 | 54.8 | 10.8 | 2.47 | 96.66 | 0.87 |
| | 52 | 5.44 | 158.6 | 33.7 | 54.4 | 11.9 | 2.43 | 96.52 | 1.05 |
| F7 | 0 | 5.53 | 99.1 | 36.1 | 52.8 | 11.1 | 1.65 | 97.56 | 0.79 |
| (10%) | 4 | 5.51 | 100.8 | 35.9 | 52.7 | 11.3 | 1.85 | 97.36 | 0.79 |
| | 8 | 5.47 | 105.0 | 35.3 | 53.8 | 10.9 | 2.03 | 97.20 | 0.77 |
| | 13 | | | 34.9 | 53.8 | 11.3 | 2.14 | 96.98 | 0.88 |
| | 26 | 5.49 | 97.2 | 35.5 | 53.1 | 11.3 | 2.43 | 96.69 | 0.88 |
| | 42 | 5.58 | 105.2 | 36.8 | 53.0 | 10.2 | 2.52 | 96.62 | 0.86 |
| | 52 | 5.48 | 102.8 | 35.9 | 52.4 | 11.6 | 2.54 | 96.55 | 0.91 |

TABLE 7
long term stability data at 25° C.

| | 25° C. | | | | | | | | |
|---|---|---|---|---|---|---|---|---|---|
| | | | | | | | SE UPLC | | |
| | | | A280 | iCE3 | | | HMWS | Mono | LMWS |
| | Week | pH | (mg/mL) | APG | Main | BPG | (%) | (%) | (%) |
| F1 | 0 | 5.51 | 100.9 | 34.7 | 54.0 | 11.3 | 1.53 | 97.69 | 0.78 |
| (10%) | 4 | 5.48 | 108.6 | 33.8 | 53.4 | 12.8 | 2.00 | 97.16 | 0.84 |
| | 8 | 5.44 | 106.5 | 34.7 | 51.4 | 13.9 | 2.23 | 96.84 | 0.93 |
| | 13 | | | 35.6 | 51.3 | 13.0 | 2.39 | 96.33 | 1.28 |
| | 26 | 5.46 | 97.5 | 39.2 | 45.9 | 14.9 | 2.63 | 95.88 | 1.49 |
| F2 | 0 | 5.51 | 127.6 | 35.4 | 53.6 | 11.0 | 1.63 | 97.59 | 0.78 |
| (12.5%) | 4 | 5.48 | 138.6 | 33.7 | 53.7 | 12.6 | 2.19 | 96.96 | 0.85 |
| | 8 | 5.43 | 132.3 | 35.4 | 51.0 | 13.6 | 2.47 | 96.60 | 0.93 |
| | 13 | | | 36.0 | 49.8 | 14.2 | 2.65 | 96.05 | 1.30 |
| | 26 | 5.46 | 128.0 | 39.8 | 45.7 | 14.6 | 2.96 | 95.59 | 1.45 |
| F3 | 0 | 5.51 | 152.3 | 34.7 | 54.4 | 10.9 | 1.76 | 97.47 | 0.77 |
| (15%) | 4 | 5.47 | 157.8 | 33.9 | 52.8 | 13.3 | 2.41 | 96.75 | 0.84 |
| | 8 | 5.44 | 156.9 | 35.5 | 51.0 | 13.5 | 2.68 | 96.46 | 0.86 |
| | 13 | | | 35.5 | 50.5 | 14.0 | 2.87 | 95.83 | 1.30 |
| | 26 | 5.46 | 142.7 | 39.1 | 46.3 | 14.6 | 3.28 | 95.25 | 1.46 |
| F4 | 0 | 5.50 | 178.1 | 34.9 | 54.0 | 11.1 | 1.88 | 97.37 | 0.75 |
| (17.5%) | 4 | 5.48 | 182.6 | 34.9 | 52.4 | 12.7 | 2.62 | 96.56 | 0.82 |
| | 8 | 5.44 | 187.3 | 35.2 | 51.5 | 13.3 | 2.96 | 96.15 | 0.89 |
| | 13 | | | 35.7 | 50.6 | 13.7 | 3.19 | 95.55 | 1.26 |
| | 26 | 5.46 | 177.0 | 39.5 | 45.5 | 15.0 | 3.66 | 94.89 | 1.45 |
| F5 | 0 | 5.50 | 207.5 | 35.5 | 53.7 | 10.8 | 2.00 | 97.26 | 0.74 |
| (20%) | 4 | 5.48 | 224.8 | 34.4 | 52.8 | 12.9 | 2.86 | 96.35 | 0.80 |
| | 8 | 5.44 | 206.3 | 35.2 | 50.8 | 14.0 | 3.25 | 95.86 | 0.89 |
| | 13 | | | 36.4 | 49.5 | 14.1 | 3.50 | 95.29 | 1.21 |
| | 26 | 5.46 | 201.5 | 39.3 | 46.1 | 14.6 | 4.04 | 94.55 | 1.41 |
| F6 | 0 | 5.52 | 151.9 | 34.3 | 54.3 | 11.4 | 1.71 | 97.53 | 0.76 |
| (15%) | 4 | 5.47 | 154.6 | 34.2 | 53.0 | 12.8 | 2.40 | 96.77 | 0.83 |
| | 8 | 5.44 | 158.6 | 35.1 | 51.0 | 13.9 | 2.70 | 96.40 | 0.90 |
| | 13 | | | 35.8 | 50.2 | 14.0 | 2.90 | 95.89 | 1.20 |
| | 26 | 5.46 | 147.9 | 40.1 | 45.4 | 14.5 | 3.35 | 95.25 | 1.40 |
| F7 | 0 | 5.53 | 99.1 | 36.1 | 52.8 | 11.1 | 1.65 | 97.56 | 0.79 |
| (10%) | 4 | 5.50 | 106.4 | 36.0 | 51.9 | 12.2 | 2.37 | 96.80 | 0.84 |
| | 8 | 5.45 | 108.7 | 37.7 | 49.5 | 12.9 | 2.70 | 96.39 | 0.91 |
| | 13 | | | 38.1 | 49.0 | 12.9 | 2.95 | 95.99 | 1.06 |
| | 26 | 5.49 | 93.9 | 44.1 | 43.5 | 12.4 | 3.35 | 95.40 | 1.25 |

TABLE 8
Stability data at 40° C.

| | 40° C. | | | | | | | | |
|---|---|---|---|---|---|---|---|---|---|
| | | | | | | | SE UPLC | | |
| | | | A280 | iCE3 | | | HMWS | Mono | LMWS |
| | Week | pH | (mg/mL) | APG | Main | BPG | (%) | (%) | (%) |
| F1 | 0 | 5.51 | 100.9 | 34.7 | 54.0 | 11.3 | 1.53 | 97.69 | 0.78 |
| (10%) | 4 | 5.48 | 110.7 | 40.4 | 42.0 | 17.7 | 3.70 | 94.24 | 2.06 |
| | 8 | 5.45 | 109.5 | 46.1 | 34.1 | 19.9 | 4.79 | 92.02 | 3.19 |
| | 13 | | | 57.0 | 23.2 | 19.8 | 7.77 | 87.87 | 4.35 |
| F2 | 0 | 5.51 | 127.6 | 35.4 | 53.6 | 11.0 | 1.63 | 97.59 | 0.78 |
| (12.5%) | 4 | 5.48 | 135.6 | 39.4 | 42.4 | 18.2 | 4.19 | 93.72 | 2.09 |
| | 8 | 5.47 | 137.3 | 49.4 | 30.9 | 19.7 | 5.53 | 91.38 | 3.09 |
| | 13 | | | 57.1 | 23.5 | 19.4 | 8.51 | 87.34 | 4.14 |
| F3 | 0 | 5.51 | 152.3 | 34.7 | 54.4 | 10.9 | 1.76 | 97.47 | 0.77 |
| (15%) | 4 | 5.48 | 157.6 | 39.6 | 42.3 | 18.1 | 4.63 | 93.40 | 1.97 |
| | 8 | 5.46 | 158.3 | 49.0 | 27.6 | 23.3 | 6.22 | 90.61 | 3.17 |
| | 13 | | | 56.7 | 23.1 | 20.2 | 9.28 | 86.73 | 3.99 |
| F4 | 0 | 5.50 | 178.1 | 34.9 | 54.0 | 11.1 | 1.88 | 97.37 | 0.75 |
| (17.5%) | 4 | 5.49 | 185.4 | 40.1 | 41.2 | 18.6 | 5.12 | 92.94 | 1.94 |
| | 8 | 5.45 | 189.2 | 46.8 | 33.3 | 19.9 | 6.97 | 90.09 | 2.95 |
| | 13 | | | 56.4 | 19.5 | 24.1 | 10.12 | 85.96 | 3.92 |
| F5 | 0 | 5.50 | 207.5 | 35.5 | 53.7 | 10.8 | 2.00 | 97.26 | 0.74 |
| (20%) | 4 | 5.48 | 206.4 | 39.8 | 41.6 | 18.6 | 5.60 | 92.56 | 1.84 |
| | 8 | 5.49 | 224.4 | 49.0 | 27.0 | 24.0 | 7.70 | 89.55 | 2.76 |
| | 13 | | | 54.6 | 21.8 | 23.6 | 11.06 | 85.16 | 3.78 |
| F6 | 0 | 5.52 | 151.9 | 34.3 | 54.3 | 11.4 | 1.71 | 97.53 | 0.76 |
| (15%) | 4 | 5.48 | 162.9 | 39.8 | 42.4 | 17.7 | 4.64 | 93.44 | 1.93 |
| | 8 | 5.47 | 166.5 | 50.0 | 31.1 | 19.0 | 6.40 | 90.72 | 2.88 |
| | 13 | | | 56.0 | 21.8 | 22.3 | 9.42 | 86.59 | 4.00 |
| F7 | 0 | 5.53 | 99.1 | 36.1 | 52.8 | 11.1 | 1.65 | 97.56 | 0.79 |
| (10%) | 4 | 5.51 | 106.0 | 44.1 | 40.1 | 15.8 | 3.79 | 94.61 | 1.60 |
| | 8 | 5.47 | 110.8 | 56.1 | 28.8 | 15.1 | 5.29 | 92.37 | 2.34 |
| | 13 | | | 65.6 | 21.4 | 13.0 | 7.47 | 89.31 | 3.22 |

TABLE 9
Viscosity

| Formulation (T0) | Viscosity (cP) |
|---|---|
| F1 | 2.74 |
| F2 | 3.94 |
| F3 | 5.8 |
| F4 | 10.09 |
| F5 | 19.65 |
| F6 | 5.89 |
| F7 | 3.55 |

### Overall Conclusion

Overall, the formulations F1 to F5 were more stable after storage, especially at 500 (T52 w) and 2500 (T26 w) conditions at the same concentration than the formulations F6 and F7. At 500 and 2500 comparable stability was observed between formulations F3 and F7. As a general observation, the higher the concentration of the antibody, the higher aggregation level, as expected. However, even at 20%, these levels of aggregation were acceptable. There was no effect of presence/absence of PS80 (formulations F3 vs F6) for the iCE data, except a slight increase of turbidity (visual assessment) without PS80 (formulation F6). Although the preferred formulation was F3 (15% of mAb1), any one of F2 (12.5% of mAb1) to F5 (20% of mAb1) were acceptable and possible backup options.

### REFERENCES

1) WO2006100679
2) WO2012146901
3) WO2013175229

1. A stable liquid formulation comprising an anti-TG2 antibody, a buffer keeping the pH between about 5.0 and 6.0, and an amino acid stabilizer.

2. The stable liquid formulation according to claim 1, wherein the buffer is a histidine buffer.

3. The stable liquid formulation according to claim 2, wherein the histidine buffer keeps the pH at or about 5.5±0.2.

4. The stable liquid formulation according to claim 1, wherein the concentration of the buffer is at or about 10 to 100 mM, preferably 20 to 80 or even preferably 40 to 60 mM.

5. The stable liquid formulation according to claim 1, wherein the amino acid stabilizer is arginine or an arginine salt and is in an amount from about 100 mM to 300 mM, preferably 110 to 250 mM or even preferably 120 to 200 mM.

6. The stable liquid formulation according to claim 5, wherein the arginine salt is arginine-HCl.

7. The stable liquid formulation according to claim 1, further optionally comprising a polysorbate surfactant.

8. The stable liquid formulation according to claim 7, wherein the concentration of the polysorbate surfactant is at or about 0.005 to 0.1% w/v, preferably at or about 0.01 to 0.05% w/v.

9. The stable liquid formulation according to claim 1, wherein the concentration of the anti-TG2 antibody is from about 50 mg/mL to about 300 mg/mL, preferably from 110 mg/mL to 250 mg/mL or even preferably from 115 mg/mL to 220 mg/mL.

10. The stable liquid formulation according to claim 1, wherein the formulation comprises:
i) about 125 mg/mL of the anti-TG2 antibody, about 50 mM histidine buffer which keep the pH at or about 5.5, about 125 mM of Arginine-HCl, and optionally about 0.02-0.05% w/v of polysorbate,
ii) about 125 mg/mL of the anti-TG2 antibody, about 50 mM histidine buffer which keep the pH at or about 5.5, about 150 mM of Arginine-HCl, and optionally about 0.02-0.05% w/v of polysorbate,
iii) about 150 mg/mL of the anti-TG2 antibody, about 50 mM histidine buffer which keep the pH at or about 5.5, about 125 mM of Arginine-HCl, and optionally about 0.02-0.05% w/v of polysorbate,
iv) about 150 mg/mL of the anti-TG2 antibody, about 50 mM histidine buffer which keep the pH at or about 5.5, about 150 mM of Arginine-HCl, and optionally about 0.02-0.05% w/v of polysorbate,
v) about 175 mg/mL of the anti-TG2 antibody, about 50 mM histidine buffer which keep the pH at or about 5.5, about 125 mM of Arginine-HCl, and optionally about 0.02-0.05% w/v of polysorbate,
vi) about 175 mg/mL of the anti-TG2 antibody, about 50 mM histidine buffer which keep the pH at or about 5.5, about 150 mM of Arginine-HCl, and optionally about 0.02-0.05% w/v of polysorbate,
vii) about 200 mg/mL of the anti-TG2 antibody, about 50 mM histidine buffer which keep the pH at or about 5.5, about 125 mM of Arginine-HCl, and optionally about 0.02-0.05% w/v of polysorbate, or
viii) about 200 mg/mL of the anti-TG2 antibody, about 50 mM histidine buffer which keep the pH at or about 5.5, about 150 mM of Arginine-HCl, and optionally about 0.02-0.05% w/v of polysorbate.

11. The stable liquid formulation according to claim 1, wherein the anti-TG2 antibody comprises:
1) a light chain variable domain having the sequence as defined in SEQ ID NO: 1 and a heavy chain variable domain having the sequence as defined in SEQ ID NO:2
2) a light chain variable domain having at least 80% identity or similarity, preferably 90% identity or similarity to the sequence as defined in SEQ ID NO: 1 and a heavy chain variable domain having at least 80% identity or similarity, preferably 90% identity or similarity to the sequence as defined in SEQ ID NO: 2.
3) a light chain having the sequence as defined in SEQ ID NO: 3 and a heavy chain having the sequence as defined in SEQ ID NO: 4; or
4) a light chain having at least 80% identity or similarity, preferably 90% identity or similarity to the sequence as defined in SEQ ID NO: 3 and a heavy chain having at least 80% identity or similarity, preferably 90% identity or similarity to the sequence as defined in SEQ ID NO: 4.

12. A method for manufacturing the stable liquid formulation according to claim 1, comprising the steps of forming a mixture of the anti-TG2 antibody together with the histidine buffer, arginine-HCl and optionally the polysorbate surfactant.

13. An article of manufacture comprising a container comprising the stable liquid formulation according to claim 1.

14. The stable liquid formulation according to claim 1, for use in therapy.

15. A method of treating a disease or disorder by administering the stable liquid formulation according to claim 1.


SEQUENCE LISTING (4 sequences)

SEQ ID NO: 1 - protein, 107 aa, synthetic construct
  REGION 1..107: note = Artificial sequence
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIK

SEQ ID NO: 2 - protein, 114 aa, synthetic construct
  REGION 1..114: note = Artificial sequence
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSS

SEQ ID NO: 3 - protein, 214 aa, synthetic construct
  REGION 1..214: note = Artificial sequence
DITMTQSPSSLSASVGDRVTITCKASQDINSYLTWFQQKPGKAPKILIYLVNRLVDGVPSRFSGSGSGQDYALTISSLQPEDFATYYCLQYDDFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 441 aa, synthetic construct
  REGION 1..441: note = Artificial sequence
EVQLLESGGGLVQPGGSLRLSCAASGFTLSTHAMSWVRQAPGKGLEWVATISSGGRSTYYPDSVKGRFTISRDNSKNTLYLQMNSLRAEDTAVYFCARLISTYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK